(19) United States
(12) Patent Application Publication
(10) Pub. No.: US 2003/0152501 A1
(43) Pub. Date: Aug. 14, 2003

(54) Enclosure for imparting scent to for hunting apparel

(76) Inventors: Rick Byrd, Springdale, MO (US); Bruce Coody, Hoover, AL (US)

Correspondence Address: STREETS & STEELE, 13831 NORTHWEST FREEWAY, SUITE 355, HOUSTON, TX, 77040, US

(21) Appl. No.: 10/076,223

(22) Filed: Feb. 14, 2002

(51) Int. Cl. A61L 9/00; A62B 7/08; A61L 2/00

(52) U.S. Cl. 422/292; 422/1; 422/5; 422/123; 422/297; 422/300; 422/306

(57) ABSTRACT

The present invention provides an enclosure for storing hunting apparel while imparting either a natural or artificial scent to the stored hunting apparel to mask the human scent, thereby improving the hunting of big game animals. The container has an apparel storage compartment and a scent source storage compartment. The container may be comprised of a rigid portable enclosure, a non-rigid portable enclosure, or a cabinet.

### FIELD OF INVENTION

The present invention relates to a container for storing and treating hunting apparel and, more particularly, to a container for imparting a scent to hunting apparel, or for reducing or masking the human scent on hunting apparel, thereby improving the hunting of game.

### BACKGROUND OF THE INVENTION

The hunting of game is an increasingly popular sport. Game, as used in this document, is meant to include animals typically characterized as deer, elk and other mammals having superior olfactory senses capable of detecting the presence of humans.

One of the most widely hunted and popular game animals is the deer. Deer and other game animals have an acute sense of smell and can detect the presence of humans and articles associated with humans from great distances. It has been estimated by experts that deer have an olfactory sensitivity greater than 4,000 times that of humans.

Many scents detectable by game animals are imparted to and emanate from hunting apparel. On a hunting trip, camouflage coats, vests, overalls and hunting pants are often worn several times between washings. Repeated exposure of a garment to the human scent concentrates human scents and exacerbates the problem of detection by game. Laundry detergents and other cleaning agents used on clothes contain chemicals and perfumes that combine with human scents to make the hunter even more easily detected by game. Dogs, fuels and other animals and materials contacted by hunters contribute to detection by game.

There are existing methods and devices for preventing detection of the hunter by game. Hunters often reduce the chance of detection by applying a covering scent on their hunting apparel to mask the human scent. Hunting apparel can be stored in enclosures designed to prevent contamination by the human scent and to mask contamination by human related articles. Scented agents can be applied to the hunter's body or to hunting apparel to absorb or mask the human scent. Knight, U.S. Pat. No. 5,776,378, discloses an enclosure for hunting apparel having a power source and a motor for turning a circulation fan, also having a perforated shelf separating two compartments otherwise connected through a duct containing a scent cartridge for imparting a selected scent to air circulated across the hunting apparel. However, the integration of a portable power source and electrically-driven motor makes the forced air circulation enclosure cumbersome to those hunters who prefer to travel light.

What is needed is a portable hunting apparel enclosure for imparting a selected scent to hunting apparel and having no heavy forced power circulation, power sources or motors. What is needed is a portable hunting apparel enclosure for imparting a selected scent to hunting apparel that can be easily transported to and assembled in the field.

It is desirable that the portable hunting apparel enclosure be constructed of a lightweight and water resistant material such as coated or waxed cardboard. It is desirable that the portable hunting apparel enclosure does not require heavy components, such as batteries or a motor. It is desirable that the hunting apparel enclosure has a compartment for storing a selected scent source for imparting a masking odor to the hunting apparel stored therein.

### SUMMARY OF THE INVENTION

The present invention, a portable hunting apparel enclosure, generally relates to an enclosure for storing and transporting hunting apparel, for imparting a natural scent to the hunting apparel stored therein, and for otherwise masking the human scent using scented agents or one or more scent absorbing mediums. The scented agent may be a natural scent source such as pine needles, leaves, dirt or even an animal hormone, or scent absorbing medium such as activated charcoal.

One embodiment of the present invention is a portable enclosure that is a field assembled compartmentalized corrugated waxed cardboard box with one or more internal apparel storage compartments and one or more compartments for receiving and storing a scent source. The enclosure may be constructed of water resistant and corrugated cardboard which can be inexpensively pre-fabricated and compactly packaged for light transport and easy field assembly. The preferred embodiment comprises an easily assembled and collapsible cardboard grid placed in the bottom of a collapsible cardboard box. The collapsible grid may support a perforated shelf in a position generally parallel to the bottom of the collapsible box and supporting the perforated shelf in a position to allow the scent emanating from the scent source to permeate to all sides of stored hunting apparel that is stored upon and supported by the perforated shelf. Alternatively, the hunting apparel may rest on the grid itself.

In one embodiment, two parallel sheets of perforated cardboard form a channel down the center of the collapsible box. A compartment formed within the channel defines the scent source storage compartment, and the space between the side of each sheet opposite the scent source compartment and the adjacent top, bottom or inside wall of the enclosure defines two apparel storage compartments. The perforations in the sheets and in the hunting apparel shelf allow the scent from the scent source to diffuse and permeate from the scent source and throughout the storage compartments of the enclosure. The top or a side of the enclosure may be securable in the closed position and openable to provide access to the hunting apparel storage compartments and to the scent source compartment. Handles may be formed into or coupled to the sides of the enclosure for facilitating portability of the enclosure once assembled.

The exterior surface of the enclosure may be adapted for absorbing sunlight energy for warming hunting apparel and the scent source stored in the enclosure. For example, the outside of the container may be a dark color, whereby setting the container with the dark side disposed towards the sun would supply sufficient heat energy absorption to raise the temperature inside the enclosure, thereby increasing the concentration of the scent in the air within the enclosure. Optionally, a small heat source, with an energy source comprising either batteries or a standard electrical outlet, may be disposed in the enclosure for increasing the temperature in the enclosure before moving the enclosure to the field and thereby increasing the scent concentration in the air surrounding the hunting apparel. The higher scent concentration increases the rate of scent absorption by the hunting apparel, which is highly desirable when hunting apparel is placed in the enclosure after use to be conditioned for the next day's use. Thus, either the small heat source powered by batteries or standard electrical provisions or a radiation absorbing media, would suffice as a heat source for this invention.

An alternative embodiment of the present invention is a non-rigid baglike enclosure shaped much like a common gym bag. In one embodiment, the enclosure is made of water resistant 600 DENIER polyester and has an internal lining made of 210 DENIER nylon coated with 2× polyurethane to provide an additional barrier to moisture. The enclosure has a carrying strap or handles coupled to the enclosure to facilitate carrying the enclosure into the field on a hunting trip. The non-rigid enclosure may comprise one or more apparel storage compartments that can be opened and closed with a zipper, with the zipper going from the front of one side of the bag, along the one side, across the back and along the other side of the bag to give maximum wide access to the apparel storage compartments within. Optionally, a small heat source, with an energy source selected from batteries or a standard electrical outlet may be disposed within the enclosure for increasing the temperature in the enclosure and thereby increasing the scent concentration in the air surrounding the hunting apparel. The heat source may be powered using a cigarette lighter or other auxiliary power source. The enclosure may be adapted for retaining heat within the enclosure after disconnect from an external heat source. The higher scent concentration from warming the scent source increases the rate of scent absorption of scent by the hunting apparel, which is highly desirable when hunting apparel is placed within the enclosure after use to be conditioned for the next day's use. Alternatively, the outside of the enclosure may be a dark color and the exterior may be designed for solar absorption so that simply setting the enclosure in the sun would supply sufficient heat energy to raise the temperature inside the enclosure, thereby increasing the concentration of the scent in the air within the enclosure. Thus, either a small heat source powered by batteries, standard electrical means, or by car cigarrette lighter adapter or through the use of dark colored coverings for the enclosure, radiation energy would suffice as a heat source for this invention.

Another alternative embodiment of this invention comprises a rigid cabinet for imparting a scent from a scent source to hunting apparel stored therein. This embodiment may be constructed of furniture quality wood and may comprise casters to facilitate moving of the cabinet enclosure on a floor. A plastic laminate interior liner retards scent from the scent source from absorbing into the porous wood from which the rigid enclosure is made. The cabinet enclosure may have double doors on the front that open to allow maximum wide access to the apparel storage compartments. A rubber strip seals around the edges of the doors at the interface where the doors meet the periphery of the cabinet. This seal helps prevent the scent from the scent source from escaping the enclosure. A laminated wall runs vertically through the interior of the cabinet to divide the cabinet into two apparel storage compartments. In one compartment of the cabinet, a bar is provided for hanging hunting apparel stored using clothes hangers. A wire basket is provided in the scent source storage compartment for holding and supporting it the scent source off of the floor or bottom of the enclosure for better air circulation around the scent source. The wire basket can be removed for easy access and replenishment. Optionally, a small electrical resistance heat source that may be powered by a conventional electrical outlet or by portable batteries, such as through a cigarette lighter or auxiliary power source in a vehicle may be disposed within the enclosure for increasing the temperature in the enclosure and thereby increasing the scent concentration in the air surrounding the hunting apparel. The higher scent concentration increases the rate of scent absorption by the hunting apparel, which is highly desirable when hunting apparel is placed in the enclosure after use to be scent-conditioned for the next day's use.

### BRIEF DESCRIPTION OF THE DRAWINGS

FIG. 1 shows a perspective view of a rigid portable enclosure for storing hunting apparel.

FIG. 2 shows an exterior perspective view of non-rigid portable enclosure for storing hunting apparel in its closed position.

FIG. 3 shows an interior perspective view of the non-rigid enclosure with the top zipper opened to expose the interior storage compartment.

FIG. 4 shows a perspective view of a rigid cabinet enclosure for storing hunting apparel.

### DETAILED DESCRIPTION OF THE INVENTION

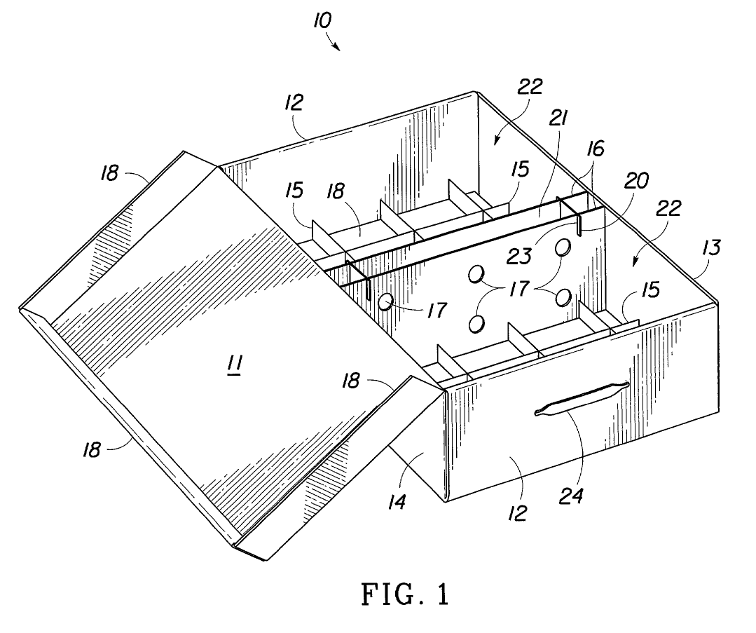

FIG. 1 shows a perspective view of the preferred embodiment of the portable enclosure 10 for storing hunting apparel. The enclosure 10 comprises a back 14, front 13, sidewalls 12, bottom 18 and top 11. In one embodiment, the enclosure and its internal components comprise water resistant or waxed corrugated cardboard. Any lightweight, rigid and water resistant material would be acceptable, for example, wax-coated cardboard or expanded foam polystyrene. A support grid 15 is placed on the bottom 18 to support the perforated shelf on which hunting apparel rests when stored in the enclosure 10 so that the scent emanating from the scent source can permeate throughout the enclosure 10 and through the hunting apparel stored therein. Alternatively, the grid 15 itself may support the hunting apparel without a perforated shelf.

The preferred embodiment of the enclosure comprises an air flow channel formed by two generally parallel dividers 16 installed near the center of the enclosure 10. Each inner divider 16 has a plurality of perforations 17 therethrough to facilitate air circulation between and through the compartments. The inner dividers 16 are given structural support at the top by inner wall supports 20 inserted through slots 23 in the dividers 16.

The area between the dividers 16 comprises a scent storage compartment 21. The scent storage compartment 21 receives the scent source, for example, a scent bar with an artificial scent or a leaf bag containing debris from the hunting site to impart a natural scent. The scent emanating from the scent source diffuses through the perforations 17 from the scent storage compartment 21 into the apparel storage compartments 22 thereby permeating through the hunting apparel and imparting the scent from the scent source to the hunting apparel. The apparel storage compartments 22 are formed between the walls of the dividers 16 and the adjacent sidewalls 12 of the enclosure 10. In one embodiment, the top 11 is hinged with the back 14 of the enclosure 10 by folding the top portion of the elongated back 14 forward to form the top 11. However, the top 11 does not have to be hinged to the back 14, and any means of either opening or removing the top 11 to provide access to the storage compartments 22 is acceptable.

It may be desirable to seal the apparel storage compartments 22 from the exterior environment to prevent the scent from inside the container 10 from escaping the enclosure. A seal also serves to maximize the concentration of a selected scent exposed to permeate the hunting apparel in the apparel storage compartments 22. In one embodiment, a flap 18 is formed around the perimeter of the top 11 and perpendicular to the top 11. The flap 18 extends over the outside edges of the sidewalls 12 and front 13 when the top 11 is closed thereby providing a seal for the enclosure 10. Handles 24 may be disposed in the side walls 12 for ease of carrying. Optionally, a small heat source with an energy source comprising batteries or a standard electrical outlet may be disposed within the enclosure for increasing the temperature within the enclosure and thereby increasing the scent concentration in the air surrounding the hunting apparel. The higher scent concentration increases the rate of scent absorption by the hunting apparel, which is highly desirable when hunting apparel is placed in the enclosure after use to be conditioned for the next day's use. Alternatively, the outside of the container may be a dark color, whereby simply setting the container outside in the sun would supply sufficient heat to raise the temperature inside the enclosure and thereby increasing the concentration of the scent in the air in the enclosure.

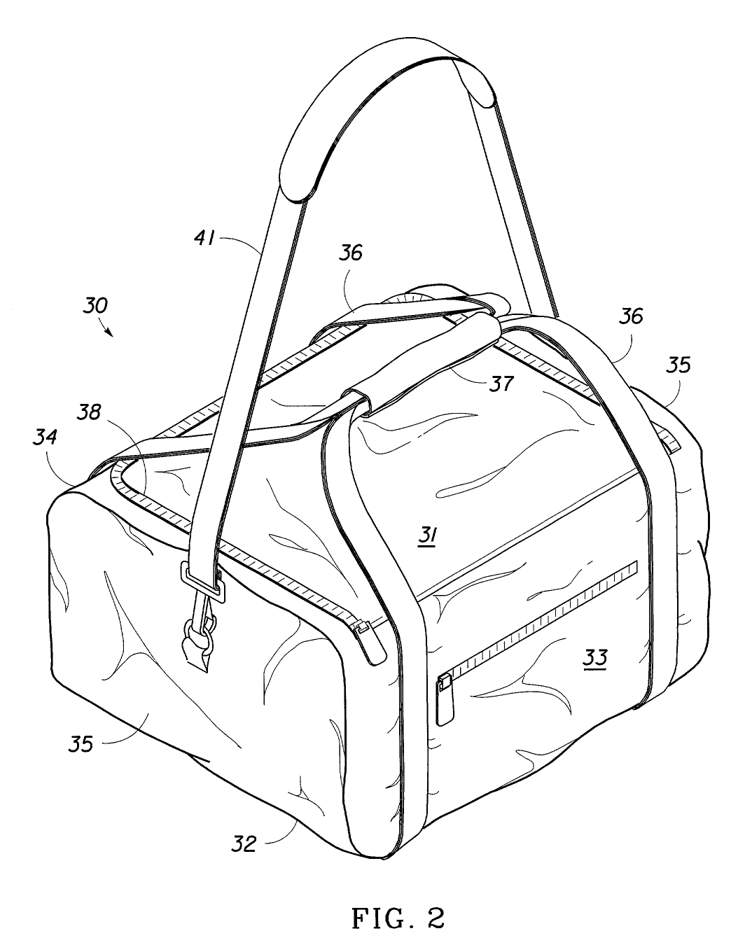

FIG. 2 shows an exterior perspective view of an alternative embodiment of the invention, a scented non-rigid enclosure 30 shaped much like a common gym bag. The enclosure 30 is constructed with a back 34, front 33, sidewalls 35, bottom 32 and top 31. In one embodiment, these components of the enclosure 30 are made of water resistant 600 DENIER polyester fabric; however, any flexible material is acceptable, including canvas, as long as the material is suitable for exposure to the elements encountered at a hunting site.

Two handle straps 36 are connected to the sides 36 on the non-rigid enclosure to facilitate carrying. These handle straps 36 are coupled to the enclosure preferably by sewing, although any means of attaching the handle straps 36 would be acceptable. Each handle strap 36 is connected at its ends to form a “U” shape and, when each handle is pulled towards the top 31, a handle clasp 37 clasps the handle straps 36 together for ease of carrying. One side of the top 31 is sewn to the front 33. The other three sides of the top 31 are held to the side walls 35 and back 34 with a zipper 38. The preferred embodiment is a self-repairing nylon zipper, but any material for the zipper 38 is acceptable. Any means for closing the top that would ensure a good seal to contain the scent within the enclosure would be acceptable, for example, VELCRO®. A shoulder strap 41 may be coupled to each sidewall 35 to facilitate ease of carrying. Optionally, a small heat source having an energy source selected from batteries or a standard electrical outlet may be disposed within the enclosure for increasing the air temperature within the enclosure and thereby increasing the scent concentration in the air surrounding the hunting apparel. The higher scent concentration increases the rate of scent absorption by the hunting apparel, which is highly desirable when hunting apparel is placed within the enclosure after use to be conditioned for the next day's use. Alternatively, the outside of the enclosure may be a dark color, whereby simply setting the enclosure in the sun would supply sufficient heat to raise the temperature inside the enclosure, thereby increasing the concentration of the scent in the air within the enclosure.

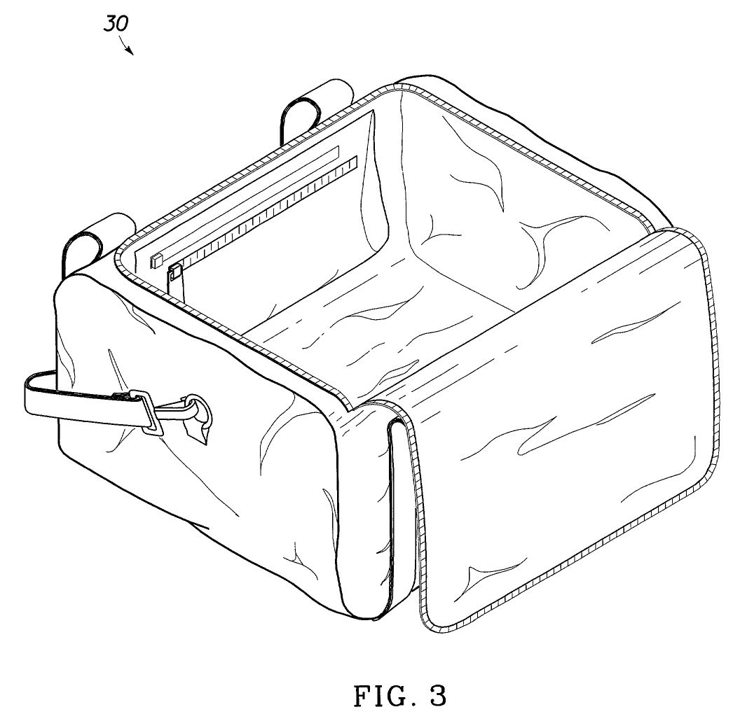

FIG. 3 shows an interior perspective view of the non-rigid enclosure 30, a non-rigid portable container for storing and imparting a scent to hunting apparel.

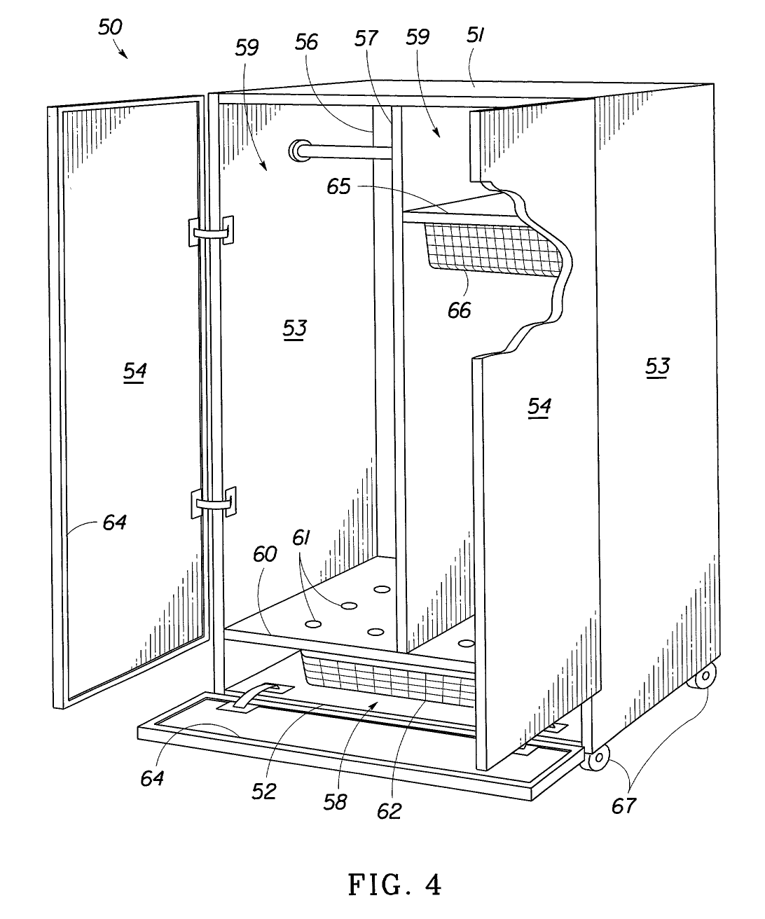

FIG. 4 is a perspective view of a cabinet-type enclosure 50 embodiment of the invention, a cabinet enclosure for storing and imparting a selected scent to hunting apparel. The enclosure 50 is constructed with a back 56, front doors 54, sidewalls 53, bottom 52 and top 51. In the preferred embodiment, these components are constructed of furniture quality oak veneer. However, any rigid material would be suitable, for example, other woods, cardboard or plastic, as long as they provide the structural support required of the enclosure 50 or have additional support members added as necessary to support the cabinet. The scent storage compartment 58 of the enclosure 50 is formed in the space between the bottom 52 and a horizontal divider 60 attached to the sidewalls 53 and back 56. Any means of attaching the horizontal divider 60 to the sidewalls 53 and back 56 is acceptable. The divider 60 has a plurality of perforations 61. An access door 64 to the scent compartment is provided. Alternatively, the doors 64 may extend downward so that, upon opening the doors 64, access to the scent storage compartment 58 is provided along with access to the apparel storage compartments 59. The scent storage compartment 58 receives the scent source, either a scent bar with an artificial scent or a leaf bag containing debris from the hunting site, to impart a natural scent to hunting apparel stored within the enclosure 50. The scent source may be stored within a wire basket 62. The scent source basket 62 may be attached to a slide to allow the wire basket 62 to slide out, or it could merely rest on the bottom 52 of the enclosure. The scent emanating from the scent source diffuses through the vent holes 61 from the scent storage compartment 58 to the apparel storage compartments 59 permeating the hunting apparel and allowing the hunting apparel to absorb the scent. The apparel storage compartments 59 are formed between the top 51, the sidewalls 53 and the divider 60. In one embodiment, a vertical apparel compartment divider 57 is attached to the back 56, divider 60 and the top 51. Two front doors 54 are connected to the cabinet enclosure with hinges to the sidewalls 53. The front doors 53 close to form the front of the enclosure 50, and open to provide wide access to the apparel storage compartment 59 and to the scent source compartment 58. A separate scent source door may be used to isolate the scent source storage compartment 58. A rubber strip 64 around the interior edge of the front doors 53 provides sealing between the edges of the top 51, bottom 52, sidewalls 53, and the apparel compartment divider 57. It is desirable to seal the apparel storage compartments 59 and the scent source storage compartment 58 from the environment to prevent the scent inside the enclosure 50 from escaping to the outside and to maximize the concentration of the scent available to permeate hunting apparel stored within the apparel storage compartments 58. The apparel compartment divider 57 defines two distinct apparel compartments 59 and also serves to minimize scent concentration loss from the container 50 when a door 54 is opened, especially if the hunter gains access to only one apparel storage compartment 58 by opening only one door 54. In one embodiment, at least one storage shelf 65 and at least one storage basket 66 attached to the shelf are included in the apparel storage compartment 59 for apparel storage. Casters 67 are attached to each bottom 52 corner to facilitate moving the cabinet 50 on a floor. Optionally, a small heat source, with an energy source of batteries or a standard electrical outlet, may be disposed within the enclosure for increasing the temperature within the enclosure and thereby increasing the scent concentration in the air surrounding the hunting apparel. The higher scent concentration increases the rate of scent absorption by the hunting apparel which is highly desirable when hunting apparel is placed in the enclosure after use to be conditioned for the next day's use.

While the foregoing is directed to the preferred embodiment of the present invention, other and further embodiments of the invention may be devised without departing from the basic scope thereof, and the scope thereof is determined by the claims that follow.

We claim:

1. A portable container for storing hunting apparel comprising:
(a) an enclosure comprising a top, bottom, and side walls;
(b) one or more apparel storage compartments formed therein;
(c) one or more scent source storage compartments formed therein in fluid communication with the one or more apparel compartments, and
(d) a heat source, wherein the increased temperature from the heat source increases the concentration of the scent in the air surrounding the apparel and wherein scent emanating from the one or more scent source compartments permeates apparel in the one or more apparel compartments.

2. The portable container of claim 1, wherein the top opens to provide access to the one or more apparel and the one or more scent source storage compartments.

3. The portable container of claim 1, wherein the top is hinged with the back by folding the cardboard at a top end of the back to form the top.

4. The portable container of claim 3, further comprising a flap formed by folding sides of the top not attached to the back, wherein the flap is perpendicular to the top, and wherein the flap provides a friction seal with each outside face of the front and the sidewalls of the container when the top is closed.

5. The portable container of claim 1, wherein the container is of any lightweight water resistant material.

6. The portable container of claim 1, wherein the container is of water resistant waxed corrugated cardboard.

7. The portable container of claim 1, further comprising lifting means.

8. The portable container of claim 7, wherein the lifting means are handles connected to the two opposing sidewalls.

9. The portable container of claim 1, further comprising a grid of interlocking strips, the grid having a top side and a bottom side, the bottom side resting on the container bottom and the top side providing support for the apparel away from the bottom of the container, wherein access for the scent permeation is provided.

10. The portable container of claim 1, further comprising two vertical, parallel dividers, each with a plurality of ventilation holes, the dividers extending across the container and dividing the container into the internal apparel storage compartment and the scent source storage compartment, wherein the plurality of ventilation holes provides fluid communication between the scent source storage compartment and the apparel storage compartments.

11. The portable container of claim 10, further comprising at least two supports for the dividers, each end of the support inserted through a slot in the divider, wherein the supports separate the dividers to form the scent source storage compartment.

12. A portable non-rigid baglike container for scenting hunting apparel comprising
(a) a non-rigid container have a top, bottom, and sidewalls;
(b) one or more apparel storage compartments formed therein; and
(c) one or more scent source storage compartments formed therein in fluid communication with the one or more apparel compartments, and
(d) a heat source, wherein the increased temperature from the heat source increases the concentration of the scent in the air surrounding the apparel and wherein scent emanating from the one or more scent source compartments permeates apparel in the one or more apparel compartments.

13. The portable non-rigid baglike container of claim 12, wherein the non-rigid container is made of a water resistant fabric-like material.

14. The portable non-rigid baglike container of claim 13, wherein the non-rigid container is made of 600 DENIER polyester.

15. The portable non-rigid baglike container of claim 12, further comprising a lining inside the non-rigid container wherein the lining covers all internal surfaces, and wherein the lining is a water resistant material.

16. The portable non-rigid baglike container of claim 14, wherein the lining is made of 210 Denier Nylon coated with 2× polyurethane.

17. The portable non-rigid baglike container of claim 13, further comprising lifting means.

18. The portable non-rigid baglike container of claim 17, wherein the lifting means are selected from handles attached to the front and the back, a shoulder strap attached to the sidewalls or combinations thereof.

19. The portable non-rigid baglike container of claim 12, wherein the top can be opened to provide access to the apparel compartment and the scent source compartment.

20. The portable non-rigid baglike container of claim 12, further comprising a means for sealing the top of the container.

21. The portable non-rigid baglike container of claim 20, wherein the means for sealing is a nylon zipper.

22. The portable non rigid bag-like container of claim 12, further comprising a means for retaining thermal energy.

23. A cabinet for storing hunting apparel comprising:
(a) a cabinet having a top, bottom, sidewalls, back, and front;
(b) one or more apparel storage compartments formed therein;
(c) one or more scent source storage compartments formed therein in fluid communication with the one or more apparel compartments, and
(d) a heat source, wherein the increased temperature from the heat source increases the concentration of the scent in the air surrounding the apparel and wherein scent emanating from the one or more scent source compartments permeates apparel in the one or more apparel compartments.

24. The cabinet of claim 23, further comprising:
two doors forming the front and providing access to the interior of the cabinet; and
a rubber strip between each door and a periphery of the cabinet, wherein the doors are attached to the sidewalls with hinges, and wherein the rubber strip seals the scent inside the cabinet when the doors are closed.

25. The cabinet of claim 23, wherein the top, bottom, sidewalls, back, and front are constructed of any rigid material having structural strength necessary to support the cabinet.

26. The cabinet of claim 23, wherein the top, bottom, sidewalls, back, and front are of any material with additional structural supports added to provide structural strength necessary to support the cabinet.

27. The cabinet of claim 23, wherein the top, bottom, sidewalls, back, and front are made of oak veneer.

28. The cabinet of claim 23, further comprising casters attached to four corners of the bottom to facilitate moving the cabinet.

29. The cabinet of claim 23, further comprising a means for containing a scent source in the scent storage compartment.

30. The cabinet of claim 29, wherein the means for containing a scent source is a wire basket on a slide attached to the bottom of the cabinet, whereby the wire basket can slide out for easy access.

31. The cabinet of claim 29, wherein the means for containing a scent source is a removable container sitting on the bottom of the cabinet.

32. The cabinet of claim 23, further comprising a vertical divider attached to the back, the top and the horizontal divider, and extending across said container, wherein the container is divided into a first apparel storage compartment and a second apparel storage compartment.

33. The cabinet of claim 32, further comprising a rod attached near a top of one side wall and near a top of the vertical divider defining the first apparel storage compartment, whereby hunting apparel can be hung from common clothes hangers.

34. The cabinet of claim 32, further comprising a plastic lamination applied to an inside surface of the top, bottom, sidewalls, doors, back, vertical divider and horizontal divider whereby said plastic lamination will prevent scent from absorbing into the oak veneer.
